(19) United States
(12) Patent Application Publication
(10) Pub. No.: US 2017/0340557 A1
(43) Pub. Date: Nov. 30, 2017

(54) SUBLINGUAL FENTANYL FORMULATIONS CONTAINING A PERMEATION ENHANCER

(71) Applicant: Insys Development Company, Inc., Chandler, AZ (US)

(72) Inventors: Ning Shan, Chandler, AZ (US); Rajesh R. Wakaskar, Chandler, AZ (US); Edwin A. Baldwin, Tempe, AZ (US); Andrew B. Schlinkert, Scottsdale, AZ (US); Min Wu, Chandler, AZ (US); Ningxin Yan, Chandler, AZ (US)

(21) Appl. No.: 15/606,125

(22) Filed: May 26, 2017

Related U.S. Application Data
(60) Provisional application No. 62/342,707, filed on May 27, 2016.

(51) Int. Cl. A61K 9/00; A61K 31/4468; A61K 47/10; A61K 47/12

(52) U.S. Cl. CPC A61K 9/006; A61K 47/12; A61K 31/4468; A61K 47/10

(57) ABSTRACT

Sublingual formulations containing fentanyl, a pharmaceutically acceptable salt or ester thereof, ethanol, propylene glycol and menthol, as well as methods of treating pain by administering the formulations of the invention to a patient in need thereof.

### FIELD OF THE INVENTION

The invention is directed to sublingual formulations containing fentanyl and a permeation enhancer at concentrations that significantly increase permeability of the formulations compared to commercial fentanyl sublingual spray formulations; and methods for treatment with the sublingual formulations.

### BACKGROUND OF THE INVENTION

Fentanyl is a μ-opioid receptor agonist with analgesic potency approximately 80-100 times that of morphine. In clinical settings, fentanyl exerts its principal pharmacologic effects on the central nervous system. Its primary actions are analgesic and sedation.

The analgesic effects of fentanyl are related to the blood level of the drug. In general, the minimum effective concentration and the concentration at which toxicity occurs rise with increasing tolerance to any and all opioids. The rate of development of tolerance may vary widely among individuals. All opioid mu-receptor agonists, including fentanyl, produce dose dependent respiratory depression. The risk of respiratory depression is typically less in patients receiving chronic opioid therapy who develop tolerance to respiratory depression and other opioid effects. Serious or fatal respiratory depression can occur, even at recommended doses, in vulnerable individuals.

Orally administered fentanyl is subject to first pass effect metabolism as upwards of 50% or more of orally administered fentanyl is not absorbed. Other forms of delivery such a parenteral, buccal, and transdermal have been utilized to decrease or avoid this first pass effect for fentanyl. Fentanyl is currently available in injectable form, as a lozenge (e.g. Actiq® (a registered trademark of Cephalon, Inc.)), as a transdermal system (e.g., Duragesic® (a registered trademark of Johnson & Johnson) 25, 50, 75, and 100 μg of fentanyl per hour) and a sublingual spray (e.g. Subsys®, a registered trademark of Insys Development Company, Inc.).

Subsys® is capable of delivering a sufficient amount of fentanyl to treat acute breakthrough pain within 5 minutes of administration. However, patients with acute breakthrough pain or other severe forms of pain such as postoperative pain would benefit from reaching peak fentanyl blood serum concentrations within the same 5 minutes after administration or sufficient blood serum concentration faster than 5 minutes after administration.

While Subsys® provides fast onset of action, there is still a need for yet faster onset to treat breakthrough cancer pain in particular. Subsys® is a patented formulation having an unique combination and concentration of ethanol and propylene glycol. See, U.S. Pat. Nos. 8,835,460; 8,486,972; 8,486,973; 8,835,459; 9,289,387; and 9,241,935. In order to achieve faster onsets, many other ingredients have been formulated and tested for many years since the Subsys® formulation was initially invented in 2007.

It has now been discovered that unique combinations and concentrations of permeation enhancers can achieve significantly better onset and pharmacokinetic parameters.

### SUMMARY OF THE INVENTION

In one aspect, the present invention is directed to a sublingual formulation comprising fentanyl or a pharmaceutically acceptable salt or ester thereof, ethanol, propylene glycol and a permeation enhancer, wherein the permeation enhancer is selected from the group consisting of: a) menthol at a concentration from 0.25% to 10% w/w; and b) a fatty acid at a concentration from 0.25% to 10% w/w, wherein w/w refers to weight by weight of the total formulation. The present invention further provides methods of treating pain by administering to a patient in need thereof a therapeutically effective amount of a composition of the present invention.

Preferably, the sublingual formulation is a spray sublingual formulation.

Preferably, the fatty acid is a medium chain fatty acid.

Preferred fatty acids are capric acid and caprylic acid.

Preferably, fentanyl or the pharmaceutically acceptable salt or ester thereof is present at a concentration from 0.1% to 2% w/w, more preferably at a concentration from 0.3% to 0.7% w/w, and even more preferably at a concentration from 0.4% to 0.6% w/w, wherein w/w refers to weight by weight of the total formulation.

The inventive formulations may have different dosage strengths. Some of the dosage strengths include, but are not limited to, 100 mcg, 200 mcg, 400 mcg, 600 mcg, 800 mcg, 1200 mcg and 1600 mcg. Further, the invention contemplates that different strengths may come in different dosage forms. For example, one strength can be administered in more than one dose.

Preferably, propylene glycol is present at a concentration from 1% to 10%, more preferably, 4% to 6%, and most preferably 5% weight by weight of the total formulation.

Preferably, ethanol is present at a concentration from 20% to 80%, more preferably 40% to 60%, and most preferably 55% weight by weight of the total formulation.

Preferably, the concentration of menthol and/or fatty acid is from 0.5% to 10% w/w, more preferably from 0.5% to 1% w/w, and even more preferably, 0.5% w/w, wherein w/w refers to weight by weight of the total formulation.

In some embodiments, the formulations of the invention comprise xylitol.

In some embodiments, the formulations of the invention have a human skin permeability coefficient of greater than 7×10⁻⁷ cm/sec, preferably greater than 1×10⁻⁶ cm/sec, and even more preferably, greater than 13×10⁻⁷ cm/sec.

In some embodiments, the formulations of the invention have flux across a human skin of greater than 0.15 μg/min/cm², preferably greater than 0.25 μg/min/cm², and even more preferably, greater than 0.35 μg/min/cm².

In one embodiment, the invention provides a sublingual spray formulation comprising fentanyl or a pharmaceutically acceptable salt or ester thereof at a concentration from 0.1% to 0.8% w/w, ethanol at a concentration from 40% to 60% w/w, propylene glycol at a concentration from 4% to 6% w/w, and menthol at a concentration from 0.25% to 10% w/w, wherein w/w refers to weight by weight of the total formulation. This formulation will preferably have a human skin permeability coefficient of greater than 7×10⁻⁷ cm/sec, and further will preferably have flux across a human skin of greater than 0.15 μg/min/cm².

The inventive formulations are stable, as determined by stability testing. Preferably, the inventive formulations contain no more than 0.06% of total impurities after four weeks at 55° C.

The inventive formulations preferably have a droplet size from 20 to 200 microns in diameter. In a more preferred embodiment, the droplet size is about 20 microns in diameter.

Preferably, the inventive formulations, when administered to humans, result in pain relief less than 5 minutes after sublingual administration, more preferably, less than 4 minutes after sublingual administration, and even more preferably, less than 3 minutes after sublingual administration.

In certain embodiments, the present invention is directed to a method of treating pain comprising administering to a patient in need thereof a therapeutically effective amount of a composition of the present invention. Preferably, the pain is breakthrough pain or postoperative pain. Even more preferably, the pain is cancer pain.

### BRIEF DESCRIPTION OF THE DRAWINGS

FIGS. 1A and 1B depict rabbit pharmacokinetic (PK) profiles of fentanyl formulations of the invention.

FIG. 2 depicts mini-pig pharmacokinetic (PK) profiles of fentanyl formulations of the invention.

### DETAILED DESCRIPTION OF THE INVENTION

The present invention is directed to a sublingual spray formulation comprising fentanyl or a pharmaceutically acceptable salt or ester thereof, ethanol, propylene glycol and a permeation enhancer, wherein the permeation enhancer is selected from the group consisting of: a) menthol at a concentration from 0.25% to 10% w/w; and b) a fatty acid at a concentration from 0.25% to 10% w/w, wherein w/w refers to weight by weight of the total formulation. The present invention further provides methods of treating pain by administering to a patient in need thereof a therapeutically effective amount of a composition of the present invention.

A surprising and unexpected effect has been found that a specific permeation enhancer selected from the group consisting of menthol and a fatty acid, wherein menthol and fatty acid are present at concentrations from 0.25% to 10% w/w, can dramatically increase permeability of the fentanyl formulation comprising ethanol and propylene glycol, yet resulting in stable formulations.

Fatty acids suitable for use in the present invention include saturated and unsaturated fatty acids. The fatty acid may be selected from the group consisting of caproic acid, enanthic acid, caprylic acid, pelargonic acid, capric acid, undecylenic acid, lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, henatriacontylic acid, heptatriacontanoic acid, octatriacontanoic acid, linolenic acid, stearidonic acid, eicosapentaenoic acid, docosahexaenoic acid, crotonic acid, myristoleic acid, palmitoleic acid, sapienic acid, oleic acid, elaidic acid, vaccenic acid, gadoleic, eicosenoic acid, erucic acid, nervonic acid, linoleic acid, eicosadienoic acid, docosadienoic acid, linolenic acid, pinolenic acid, eleostearic acid, mead acid, dihomo-linolenic acid, eicosatrienoic acid, stearidonic acid, arachidonic acid, eicosatetraenoic acid, adrenic acid, bosseopentaenoic acid, eicosapentaenoic acid, ozubondo acid, sardine acid, tetracosanolpentaenoic acid, docosahexaenoic acid and herring acid.

In a preferred embodiment the fatty acid is a medium chain fatty acid.

Medium chain fatty acids suitable for use in the present invention include, but are not limited to, caproic acid, enanthic acid, caprylic acid, pelargonic acid, capric acid, undecylenic acid, lauric acid, myristic acid and palmitic acid.

In a more preferred embodiment the fatty acid is selected from capric acid, caprylic acid and lauric acid.

The discovered enhanced permeability was very surprising and unexpected. International Patent Application Publication WO 2007/007059 to Sosei R&D Ltd. (“Sosei”) discloses that “[i]f the amount [of menthol] is too high, and especially if it is more than 0.5%, crystals may form on scale-up.” Sosei, page 1, lines 12-14. Accordingly, Sosei specifically taught away from sublingual spray formulations of fentanyl containing more than 0.5% w/w and directed skilled artisans to sublingual spray formulations of fentanyl with menthol content of less than 0.25%. Id., page 1, lines 15-18.

However, the present invention directly contradicts the teachings of Sosei and provides fentanyl sublingual formulations wherein the content of menthol is at least 0.25% w/w, and preferably at least 0.5% w/w.

The present invention also provides fentanyl sublingual formulations comprising a fatty acid, which is preferably capric acid or caprylic acid, wherein the content of the fatty acid is at least 0.25% w/w, and preferably at least 0.5% w/w. It was also very surprising and unexpected that the fatty acid, at the claimed concentrations, will result in a significant increase in permeability of sublingual fentanyl formulations.

The simplest and most prevalent administration route for pharmacologic agents is by mouth. To use this method, a pharmacological agent is typically incorporated into a tablet, a capsule, or into a liquid base. Oral administration of a drug is extremely convenient, and for many drugs, it will continue to be the method of choice.

Absorption of a drug into the bloodstream after swallowing a tablet may vary from patient to patient. The absorption of the drug is typically dependent upon the movement from the stomach to the small and large intestines and the effects of secretions from these organs. Further, with the oral administration of a drug such as fentanyl to a patient, as the fentanyl enters the patient's bloodstream through the intestines and passes through the patient's liver before distribution throughout the body, upwards of fifty percent or more of the fentanyl may be removed from the patient's bloodstream. This “first pass effect” results in the oral route of administration being impractical for fentanyl.

Absorption of fentanyl or a pharmaceutically acceptable salt thereof into the bloodstream following oral administration is significantly reduced by the first pass effect. Therefore, the oral route of administration is impractical for fentanyl. Other forms of delivery such a parenteral, buccal, and transdermal delivery have been utilized to decrease or avoid this first pass effect for fentanyl. However, these other forms of delivery have certain disadvantages associated with them. For example, parenteral administration requires injection using a syringe and needle, and may lead to necrosis that can accompany intramuscular (i.m.) administration of drugs; Actiq® (a registered trademark of Cephalon, Inc.) , a transmucosal fentanyl citrate lozenge formulation requires the patient to constantly suck on the lozenge which is attached to a handle (similar to a lollipop) in order to obtain effective pain relief; and Duragesic® (a registered trademark of Johnson & Johnson), a transdermal fentanyl delivery device, is suitable for the management of chronic pain, but is not indicated for acute or breakthrough pain.

The oral cavity offers a simple, painless method of opioid analgesic administration. Within the oral cavity, there are three generally recognized routes of administration of an active agent, namely local, buccal and sublingual.

Local delivery is mainly limited to applications regarding disruptions occurring within the oral cavity itself, such as a canker sore.

The buccal mucosa area encompasses the mucosal membranes of the inner lining of the cheeks. The buccal mucosa is however, less permeable than the sublingual area. One of the major disadvantages associated with buccal mucosa delivery of an active agent has been the relatively low passage of active agents across the mucosal epithelium, thereby resulting in low agent bioavailability, which translates into a substantial loss of usable active agent within each dosage.

Sublingual delivery is achieved through the mucosal membranes lining the floor of the mouth. Because of the high permeability and the rich blood supply, transport via the sublingual route results in a rapid onset of action, providing a delivery route appropriate for highly permeable drugs with short delivery period requirements and an infrequent dosing regimen.

The sublingual formulations of the present invention are useful in the treatment of moderate to severe pain. Preferably the sublingual formulations of the present invention are useful for the treatment of breakthrough pain, and even more preferably, for the treatment of cancer pain. For example, the formulations of the present invention are preferably suitable for a patient receiving chronic pain therapy who experiences breakthrough pain and is in need of acute pain relief.

The sublingual formulations of the present invention may be used to alleviate pain from many causes, including but not limited to shock, limb amputation, severe chemical or thermal burn injury, sprains, ligament tears, fractures, wounds and other tissue injuries, dental surgery, procedures and maladies, labor and delivery, during physical therapy, post operative pain, radiation poisoning, cancer, acquired immunodeficiency syndrome (AIDS), epidural (or peridural) fibrosis, back surgery and laminectomy, sciatica, painful sickle cell crisis, arthritis, autoimmune disease, intractable bladder pain, and the like. Sublingual administration of the formulations of fentanyl, a pharmaceutically acceptable salt thereof, or derivative thereof, of the present invention is also preferably amenable to hospice use, particularly hospices that specialize in the care of cancer and AIDS patients.

In certain preferred embodiments, the sublingual administration of the inventive formulations can relieve or alleviate episodes of acute breakthrough pain that can occur in a chronic pain condition. The inventive formulations can also be used as an adjunct therapy to a conventional treatment regimen for a chronic pain condition to alleviate breakthrough pain. In certain embodiments, the inventive formulations can be used as an anesthetic premedication, for the induction of anesthesia, for use as a sedative and/or for the treatment of anxiety.

The inventive formulations may be particularly beneficial in the patient with cancer who is unable to tolerate oral administration because of nausea and vomiting, dysphagia as a result of disease, or parenteral administration because of decreased venous access, emaciation, or coagulation defects. The inventive formulations preferably have potential advantages of even greater ease of use and rapid onset of pain relief action than existing fentanyl sublingual spray formulations.

The inventive formulations can preferably be delivered by sublingual spray pumps.

The sublingual administration of fentanyl, a pharmaceutically acceptable salt or an ester thereof, is advantageous over other forms of administration in that it does not require injection using a syringe and needle, it avoids necrosis that can accompany i.m. administration of drugs, and it avoids the need to constantly suck on a lozenge or lollipop. Preferably, the inventive formulations are suitable for self administration.

### Definitions

As used herein, all numerical values relating to amounts, weights, and the like, are defined as “about” each particular value, that is, plus or minus 10%. For example, the phrase “10% w/w” is to be understood as “9% to 11% w/w.” Therefore, amounts within 10% of the claimed value are encompassed by the scope of the claims.

As used herein “% w/w” refers to the weight percent by weight of the total formulation.

As used herein “% w/v” refers to the weight percent by volume of the total formulation.

As used herein the term “effective amount” refers to the amount necessary to treat a patient in need thereof.

As used herein the term “patient” refers to, but is not limited to, a person that is being treated for pain or another affliction or disease that can be treated with fentanyl.

As used herein the term “breakthrough pain” refers to a pain that exceeds a threshold in a patient which causes cognizable discomfort wherein the pain experienced by the patient is otherwise typically controlled e.g., by chronic analgesic therapy, and tolerated. For example, pain related to medical illnesses, such as cancer, typically fluctuates, and patients often report the experience of cognizable discomfort (e.g., breakthrough pain).

As used herein the term “pharmaceutically acceptable” refers to ingredients that are not biologically or otherwise undesirable in a sublingual dosage form.

Pharmaceutically acceptable salts that can be used in accordance with the current invention include but are not limited to hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts.

### Compositions of the Invention

Fentanyl used in the present invention may be in the form of an acid, free base or salt. Derivatives of fentanyl suitable for use in the present invention include, but are not limited to, sufentanil, carfentanil, lofentanil, alfentanil, or the like. A preferred concentration range of fentanyl suitable for the present invention is from 0.1% to 0.8% w/w, more preferably from 0.3 to 0.7% w/w, and even more preferably from 0.4% to 0.6% w/w.

The inventive formulations include ethanol and propylene glycol. Ethanol is preferably dehydrated.

Ethanol is present preferably at concentration from 40% to 60% w/w; and propylene glycol is present preferably at a concentration from 4% to 6% w/w.

The formulations of the invention can include additional solvents, which include, but are not limited to, methanol, propyl alcohol, butyl alcohol, glycerol, butylene glycol, polyethylene glycols such as polyethylene glycol (“PEG”) 200 and PEG 400 and the like. Mixtures of any of the aforementioned solvents may be used. In certain embodiments, the solvent is a non-polar hydrocarbon, preferably a C₇₋₁₈ hydrocarbon of a linear or branched configuration, its alcohols, fatty acid esters, and triglycerides, such as miglyol.

Menthol, capric acid and/or caprylic acid are present at concentrations from 0.25% to 10% w/w, preferably, from 0.5% to 10% w/w, more preferably, from 0.5% to 1% w/w, and even more preferably at 0.5% w/w.

However, the inventive formulations may include additional permeation enhancers which include, but are not limited to, limonene, carvone, transcutol, oleic acid, triacetin, Tween® 80 (polysorbate 80, Tween is a registered trademark of Croda Americas LLC), Kolliphor® EL (polyoxyl 35 hydrogenated castor oil, Kolliphor is a registered trademark of BASF SE), polyvinylpyrrolidone (“PVP”), Labrasol® (caprylocaproyl macrogol-8 glyceride, Labrasol is a registered trademark of Gattefosse SAS), sodium glycocholate, sodium lauryl sulphate, sodium taurocholate, sodium deoxycholate, triethyl citrate, mannitol, disodium laureth sulfosuccinate, N-[8-(2-hydroxybenzoyl) amino]caprylate, n-dodecyl β-D-maltoside, glyceryl monosterate, sodium caprate, and dodecyl dimethyl amino propionate.

Sweetening agents suitable for use in the present invention include, but are not limited to, sucralose, aspartame, mannitol, saccharin, xylitol, and the like. In certain preferred embodiments, the sweetening agent is xylitol. A preferred concentration range of sweetening agents suitable for the present invention is from 0.001% to 10% w/w, preferably in an amount from 0.01% to 5% w/w, and even more preferably 3% w/w.

The inventive formulations are stable, as determined by stability testing. Preferably, the inventive formulations contain no more than 0.06% of total impurities after four weeks at 55° C.

The inventive formulations are preferably aqueous, and preferably, sprays.

The inventive formulations preferably have a droplet size from 20 to 200 microns in diameter. In a more preferred embodiment, the droplet size is about 20 microns in diameter.

The inventive formulations may have different dosage strengths. Some of the dosages include, but are not limited to, 100 mcg, 200 mcg, 400 mcg, 600 mcg, 800 mcg, 1200 mcg and 1600 mcg. The invention contemplates that different strengths may be administered through different dosages. For example, one strength may be administered through more than one dosages.

In certain embodiments, the invention provides fentanyl sublingual spray formulations exhibiting a mean maximum plasma concentration (Cₘₐₓ) of about 1.0 ng/ml ±1.6 based on a sublingual dose of about 200 mcg fentanyl when administered sublingually to Yucatan mini-pigs.

In certain embodiments, the invention provides fentanyl sublingual spray formulations providing a mean time to maximum plasma concentration (Tₘₐₓ) of about 15 minutes when administered sublingually to Yucatan mini-pigs.

In certain embodiments, the invention provides fentanyl sublingual spray formulations providing an area under the plasma concentration time curve to 24 hours (AUC₀₋₂₄ₕ) of about 90.8±1.6 ng*min/mL when administered sublingually to Yucatan mini-pigs.

### PREFERRED EMBODIMENTS

In one preferred embodiment, the present invention is directed to a sublingual spray formulation comprising 0.44% w/w fentanyl base, 54.5% ethanol (“EtOH”), 4.95% w/w propylene glycol (“PG”), 0.5% w/w menthol, 2.97% xylitol and 36.64% water. This formulation preferably has flux of 0.38 μg/min/cm² and preferably has permeability coefficient of 15.7±2.62×10⁻⁷ cm/sec.

In another preferred embodiment, the present invention is directed to a sublingual spray formulation comprising 0.44% w/w fentanyl base, 54.5% ethanol (“EtOH”), 4.95% w/w propylene glycol (“PG”), 1% w/w menthol, 2.97% xylitol and 36.14% water. This formulation preferably has flux of 0.22 μg/min/cm² and preferably has permeability coefficient of 9.19±1.49×10⁻⁷ cm/sec.

In another preferred embodiment, the present invention is directed to a sublingual spray formulation comprising 0.44% w/w fentanyl base, 54.5% ethanol (“EtOH”), 4.95% w/w propylene glycol (“PG”), 0.05% w/w menthol, 0.5% capric acid, 2.97% xylitol and 36.59% water. This formulation preferably has flux of 0.53 μg/min/cm² and preferably has permeability coefficient of 22.2±6.83×10⁻⁷ cm/sec.

In another preferred embodiment, the present invention is directed to a sublingual spray formulation comprising 0.44% w/w fentanyl base, 54.5% ethanol (“EtOH”), 4.95% w/w propylene glycol (“PG”), 0.05% w/w menthol, 0.5% caprylic acid, 2.97% xylitol and 36.59% water. This formulation preferably has flux of 0.40 μg/min/cm² and preferably has permeability coefficient of 16.7±3.02×10⁻⁷ cm/sec.

The following Examples are provided solely for illustrative purposes and are not meant to limit the invention in any way.

### EXAMPLE 1

### Preparation of Novel Fentanyl Formulations

Sublingual spray formulations were created by first degassing ethanol and USP purified water, separately. Soluble excipients were then dissolved in either ethanol or purified water based on their solubility. Next, the above solutions were combined. Fentanyl was added to the final solution and mixed until dissolved.

TABLE 1
Fentanyl Sublingual Spray Formulations for Stability Studies and
Droplet testing.

| | Formulation | | | | | |
|---|---|---|---|---|---|---|
| | #1 | Formulation | Formulation | Formulation | Formulation | Formulation |
| Component | (Subsys ®) | #2 | #3 | #4 | #5 | #6 |
| Fentanyl Base | 0.4395 | 0.4395 | 0.4395 | 0.4395 | 0.4395 | 0.4395 |
| Dehydrated | 54.5 | 54.5 | 54.5 | 54.5 | 54.5 | 54.5 |
| Alcohol | | | | | | |
| Propylene | 4.945 | 4.945 | 4.945 | 4.945 | 4.945 | 4.945 |
| Glycol | | | | | | |
| L-Menthol | 0.05 | 0.05 | 0.05 | 0.05 | 0.5 | 0.5 |
| Xylitol | 2.967 | 2.967 | 2.967 | 2.967 | 2.967 | 2.967 |
| Capric acid | 0 | 0 | 0.5 | 0 | 0 | 0 |
| (C10) | | | | | | |
| Caprylic Acid | 0 | 0 | 0 | 0.5 | 0 | 2 |
| (C8) | | | | | | |
| Sodium | 0 | 0.2 | 0 | 0 | 0 | 0 |
| Deoxycholate | | | | | | |
| Benzalkonium | 0 | 0 | 0 | 0 | 0 | 0.01 |
| Chloride | | | | | | |
| Purified water | 37.0985 | 36.8985 | 36.5985 | 36.5985 | 36.6485 | 34.6385 |
| Total | 100 | 100 | 100 | 100 | 100 | 100 |

Values = % w/w

### EXAMPLE 2

### Stability Testing of Fentanyl Formulations

The formulations listed in Table 1 were subjected to stability testing at 55° C.±2° C., for eight weeks. Those formulations were also subjected to stability testing at 25° C/60%±5% RH and 40° C. under 75%±5% relative humidity. The stability data at 25° C./60%±5% RH were collected at zero, four, eight weeks, and three months. The stability data at 40° C./75%±5% were collected at zero, two, four, eight weeks, three and four months. The stability data at 55° C. were collected at zero, two, four six and eight weeks. Assay and impurities were detected using high performance liquid chromatography with an ultraviolet detector. The assay was performed at 230 nm and indicated as a percentage of initial concentration. For all impurities, analysis was performed at 230 nm and expressed as a % area. The amount of each particular impurity is listed in Tables 2A to 2L as a percentage of the area of each formulation along with amount of total impurities. “BQL” refers to “Below Quantifiable Limit” and “ND” refers to “Not Detected.”

The fentanyl formulations of the present invention contained no more than 0.06% of total impurities after four weeks at 55° C.±2° C.

TABLES 2A to 2L
Stability Data for Sublingual Fentanyl Spray Formulations
stored at 55 ± 2° C., 40 ± 2° C./75% ± 5% RH and 25° C. ± 2° C./60% ± 5% RH
2A. Stability Data for Formulation #2 (0.2% Sodium Deoxycholate) at 55° C. ± 2° C.

| | SOP | | | T = | T = 4 | T = 6 | |
|---|---|---|---|---|---|---|---|
| | Limit | RRT | T = 0 | 2 wk | wk | wk | T = 8 wk |
| Physical | Clear | | Clear | Clear | Clear | Clear | Clear |
| appearance | and | | | | | | |
| | Colorless | | | | | | |
| Assay | 90-110% | | 100.00% | 98.51% | 100.75% | 101.24% | 101.24% |
| Propioanilide | 0.50% | 0.39- | ND | BQL | BQL | BQL | BQL |
| | | 0.41 | | | | | |
| Acetyl | 0.30% | 0.56 | ND | 0.06 | ND | ND | ND |
| Fentanyl | | | | | | | |
| N-Amine | 0.30% | 0.70 | ND | ND | ND | ND | ND |
| Total | 1.50% | | 0.00% | 0.06% | 0.00% | 0.00% | 0.00% |
| Impurities | | | | | | | |

2B. Stability Data for Formulation #3 (0.5% Capric Acid) at 55° C. ± 2° C.

| | SOP | | | | | | |
|---|---|---|---|---|---|---|---|
| | Limit | RRT | T = 0 | T = 2 wk | T = 4 wk | T = 6 wk | T = 8 wk |
| Physical | Clear | | Clear | Clear | Clear | Clear | Clear |
| appearance | and | | | | | | |
| | Colorless | | | | | | |
| Assay | 90-110% | | 100.00% | 100.00% | 101.27% | 101.02% | 101.53% |
| Propioanilide | 0.50% | 0.39- | ND | BQL | BQL | BQL | BQL |
| | | 0.41 | | | | | |
| Acetyl | 0.30% | 0.56 | ND | ND | 0.06 | ND | BQL |
| Fentanyl | | | | | | | |
| N-Amine | 0.30% | 0.70 | ND | ND | ND | ND | ND |
| Total | 1.50% | | 0.00% | 0.00% | 0.06% | 0.00% | 0.00% |
| Impurities | | | | | | | |

2C. Stability Data for Formulation #4 (0.5% Caprylic Acid) at 55° C. ± 2° C.

| | SOP Limit | RRT | T = 0 | T = 2 wk | T = 4 wk | T = 6 wk | T = 8 wk |
|---|---|---|---|---|---|---|---|
| Physical | Clear and | | Clear | Clear | Clear | Clear | Clear |
| appearance | Colorless | | | | | | |
| Assay | 90-110% | | 100.00% | 100.25% | 100.75% | 101.00% | 101.75% |
| Propioanilide | 0.50% | 0.39-0.41 | ND | ND | BQL | BQL | BQL |
| Acetyl | 0.30% | 0.56 | ND | ND | BQL | ND | BQL |
| Fentanyl | | | | | | | |
| N-Amine | 0.30% | 0.70 | ND | ND | ND | ND | ND |
| Total | 1.50% | | 0.00% | 0.00% | 0.00% | 0.00% | 0.00% |
| Impurities | | | | | | | |

2D. Stability Data for Formulation #5 (0.5% Menthol) at 55° C. ± 2° C.

| | SOP | | | | | | |
|---|---|---|---|---|---|---|---|
| | Limit | RRT | T = 0 | T = 2 wk | T = 4 wk | T = 6 wk | T = 8 wk |
| Physical | Clear | | Clear | Clear | Clear | Clear | Clear |
| appearance | and | | | | | | |
| | Colorless | | | | | | |
| Assay | 90-110% | | 100.00% | 100.00% | 101.77% | 101.01% | 101.52% |
| Propioanilide | 0.50% | 0.39-0.41 | ND | BQL | BQL | BQL | BQL |
| Acetyl | 0.30% | 0.56 | ND | BQL | ND | BQL | BQL |
| Fentanyl | | | | | | | |
| N-Amine | 0.30% | 0.70 | ND | ND | ND | ND | ND |
| Unspecified | 0.10% | 0.62 | ND | ND | ND | BQL | ND |
| Impurities | | | | | | | |
| Total | 1.50% | | 0.00 | 0.00 | 0.00 | 0.00 | 0.00 |
| Impurities | | | | | | | |
| (%) | | | | | | | |

2E. Stability Data for Formulation #2 (0.2% Sodium Deoxycholate) at 40° C. ± 2° C./
75% ± 5% RH

| | | | | T = | T = | T = | T = | T = |
|---|---|---|---|---|---|---|---|---|
| | SOP Limit | RRT | T = 0 | 2 wk | 4 wk | 8 wk | 3 month | 4 month |
| Physical | Clear and | | Clear | Clear | Clear | Clear | Clear | Clear |
| appearance | Colorless | | | | | | | |
| Assay | 90-110% | | 100.00% | 99.50% | 100.50% | 100.00% | 99.75% | 99.00% |
| Propioanilide | 0.50% | 0.39- | ND | BQL | BQL | BQL | BQL | BQL |
| | | 0.41 | | | | | | |
| Acetyl | 0.30% | 0.56 | ND | BQL | ND | ND | BQL | BQL |
| Fentanyl | | | | | | | | |
| N-Amine | 0.30% | 0.70 | ND | ND | ND | ND | ND | ND |
| Total | 1.50% | | 0.00% | 0.00% | 0.00% | 0.00% | 0.00% | 0.00% |
| Impurities | | | | | | | | |

2F. Stability Data for Formulation #3 (0.5% Capric Acid) at 40° C. ± 2° C./75% ± 5% RH

| | SOP | | | T = | T = | T = | T = | T = |
|---|---|---|---|---|---|---|---|---|
| | Limit | RRT | T = 0 | 2 wk | 4 wk | 8 wk | 3 month | 4 month |
| Physical | Clear | | Clear | Clear | Clear | Clear | Clear | Clear |
| appearance | and | | | | | | | |
| | Colorless | | | | | | | |
| Assay | 90- | | 100.00% | 100.00% | 101.78% | 100.76% | 100.00% | 100.51% |
| | 110% | | | | | | | |
| Propioanilide | 0.50% | 0.39- | ND | ND | BQL | BQL | BQL | BQL |
| | | 0.41 | | | | | | |
| Acetyl | 0.30% | 0.56 | ND | ND | ND | ND | BQL | BQL |
| Fentanyl | | | | | | | | |
| N-Amine | 0.30% | 0.70 | ND | ND | ND | ND | ND | ND |
| Specified | 0.30% | 0.38 | ND | ND | ND | ND | ND | ND |
| Unknown | | 0.44 | ND | ND | ND | ND | ND | ND |
| Impurities | | 0.90 | ND | ND | ND | ND | ND | ND |
| | | 1.10 | ND | ND | ND | ND | ND | ND |
| | | 1.55 | ND | ND | ND | ND | ND | ND |
| | | 1.75 | ND | ND | ND | ND | ND | ND |
| | | 2.10 | ND | ND | ND | ND | ND | ND |
| Total | 1.50% | | 0.00% | 0.00% | 0.00% | 0.00% | 0.00% | 0.00% |
| Impurities | | | | | | | | |

2G. Stability Data for Formulation #4 (0.5% Caprylic Acid) at 40° C. ± 2° C./75% ± 5% RH

| | SOP | | | T = | T = | T = | T = | T = |
|---|---|---|---|---|---|---|---|---|
| | Limit | RRT | T = 0 | 2 wk | 4 wk | 8 wk | 3 month | 4 month |
| Physical | Clear | | Clear | Clear | Clear | Clear | Clear | Clear |
| appearance | and | | | | | | | |
| | Colorless | | | | | | | |
| Assay | 90- | | 100.00% | 99.50% | 100.75% | 101.00% | 99.75% | 100.50% |
| | 110% | | | | | | | |
| Propioanilide | 0.50% | 0.39- | ND | ND | BQL | BQL | BQL | BQL |
| | | 0.41 | | | | | | |
| Acetyl | 0.30% | 0.56 | ND | ND | BQL | ND | BQL | BQL |
| Fentanyl | | | | | | | | |
| N-Amine | 0.30% | 0.70 | ND | ND | ND | ND | ND | ND |
| Total | 1.50% | | 0.00% | 0.00% | 0.00% | 0.00% | 0.00% | 0.00% |
| Impurities | | | | | | | | |

2H. Stability Data for Formulation #5 (0.5% Menthol) at 40° C. ± 2° C./75% ± 5% RH

| | | | | T = | T = | T = | T = | T = |
|---|---|---|---|---|---|---|---|---|
| | SOP Limit | RRT | T = 0 | 2 wk | 4 wk | 8 wk | 3 month | 4 month |
| Physical | Clear and | | Clear | Clear | Clear | Clear | Clear | Clear |
| appearance | Colorless | | | | | | | |
| Assay | 90-110% | | 100.00% | 99.24% | 97.97% | 100.25% | 100.25% | 100.00% |
| Propioanilide | 0.50% | 0.39- | ND | BQL | BQL | BQL | BQL | BQL |
| | | 0.41 | | | | | | |
| Acetyl | 0.30% | 0.56 | ND | BQL | BQL | BQL | BQL | BQL |
| Fentanyl | | | | | | | | |
| N-Amine | 0.30% | 0.70 | ND | ND | ND | ND | ND | ND |
| Total | 1.50% | | 0.00% | 0.00% | 0.00% | 0.00% | 0.00% | 0.00% |
| Impurities | | | | | | | | |

2I. Stability Data for Formulation #2 (0.2% sodium Deoxycholate) at 25° C. ±
2° C./60% ± 5% RH

| | SOP | | | | | T = |
|---|---|---|---|---|---|---|
| | Limit | RRT | T = 0 | T = 4 wk | T = 8 wk | 3 month |
| Physical | Clear | | Clear | Clear | Clear | Clear |
| appearance | and | | | | | |
| | Colorless | | | | | |
| Assay | 90-110% | | 100.00% | 100.25% | 99.75% | 99.50% |
| Propioanilide | 0.50% | 0.39- | ND | BQL | BQL | BQL |
| | | 0.41 | | | | |
| Acetyl | 0.30% | 0.56 | ND | ND | ND | BQL |
| Fentanyl | | | | | | |
| N-Amine | 0.30% | 0.70 | ND | ND | ND | ND |
| Total | 1.50% | | 0.00% | 0.00% | 0.00% | 0.00% |
| Impurities | | | | | | |

2J. Stability Data for Formulation #3 (0.5% Capric Acid) at 25° C. ± 2° C./
60% ± 5% RH

| | |
|---|---|
| SOP | T = |

| | Limit | RRT | T = 0 | T = 4 wk | T = 8 wk | 3 month |
|---|---|---|---|---|---|---|
| Physical | Clear | | Clear | Clear | Clear | Clear |
| appearance | and | | | | | |
| | Colorless | | | | | |
| Assay | 90-110% | | 100.00% | 101.53% | 102.29% | 100.25% |
| Propioanilide | 0.50% | 0.39- | ND | ND | ND | ND |
| | | 0.41 | | | | |
| Acetyl | 0.30% | 0.56 | ND | BQL | BQL | ND |
| Fentanyl | | | | | | |
| N-Amine | 0.30% | 0.70 | ND | ND | ND | ND |
| Total | 1.50% | | 0.00% | 0.00% | 0.00% | 0.00% |
| Impurities | | | | | | |

2K. Stability Data for Formulation #4 (0.5% Caprylic Acid) at 25° C. ± 2° C./
60% ± 5% RH

| | SOP | | | T = | | T = |
|---|---|---|---|---|---|---|
| | Limit | RRT | T = 0 | 4 wk | T = 8 wk | 3 month |
| Physical | Clear | | Clear | Clear | Clear | Clear |
| appearance | and | | | | | |
| | Colorless | | | | | |
| Assay | 90-110% | | 100.00% | 99.75% | 100.75% | 100.00% |
| Propioanilide | 0.50% | 0.39- | ND | ND | ND | ND |
| | | 0.41 | | | | |
| Acetyl | 0.30% | 0.56 | ND | BQL | ND | ND |
| Fentanyl | | | | | | |
| N-Amine | 0.30% | 0.7 | ND | ND | ND | ND |
| Total | 1.50% | | 0.00% | 0.00% | 0.00% | 0.00% |
| Impurities | | | | | | |

2L. Stability Data for Formulation #5 (0.5% Menthol) at 25° C. ± 2° C./
60% ± 5% RH

| | SOP | | | | | T = |
|---|---|---|---|---|---|---|
| | Limit | RRT | T = 0 | T = 4 wk | T = 8 wk | 3 month |
| Physical | Clear | | Clear | Clear | Clear | Clear |
| appearance | and | | | | | |
| | Colorless | | | | | |
| Assay | 90-110% | | 100.00% | 100.76% | 100.51% | 98.99% |
| Propioanilide | 0.50% | 0.39- | ND | BQL | BQL | BQL |
| | | 0.41 | | | | |
| Acetyl | 0.30% | 0.56 | ND | BQL | BQL | ND |
| Fentanyl | | | | | | |
| N-Amine | 0.30% | 0.7 | ND | ND | ND | ND |
| Total | 1.50% | | 0.00% | 0.00% | 0.00% | 0.00% |
| Impurities | | | | | | |

### EXAMPLE 3

### Droplet Testing

In order to determine the spray profiles for all formulations, it was subjected to standardized droplet testing. The optimal particle size for sublingual spray droplets is from 20 to about 200 microns in diameter. It is desirable for the formulation to have droplet sizes near 20 because this increases the surface area and increased surface area exposure is one factor that contributes to a high bioavailability. Sublingual formulations should be able to maintain a consistent droplet size throughout its shelf life.

Droplet analysis was conducted using standard laser analysis procedures known by those of skill in the art. Droplet size distribution (Dv10, Dv50, Dv90, and Span were tested at two distances, 3 cm and 6 cm). Dv10 refers to droplet size for which 10% of the total volume is obtained; Dv50 refers to droplet size for which 50% of the total volume is obtained; Dv90 refers to droplet size for which 90% of the total volume is obtained; Span refers to distribution span (Dv90−Dv10)/Dv50; % RSD refers to the percent relative standard deviation. The results of these tests can be seen below in Tables 3 to 12. Applicant found during testing that formulations of the present invention yielded desirable droplet sizes for sublingual administration. The testing also revealed that the formulation dose remains consistent when administered with a spray pump.

As can be seen in Tables 3 to 12, all formulations of the present invention provided excellent plume geometry and spray patterns.

TABLE 3
A. Particle Size of Fentanyl Spray Formulation #2, at 3 cm

| | | Particle Size | | | | |
|---|---|---|---|---|---|---|
| | | DV(10) | DV(50) | DV(90) | | |
| Formulation #2 | | μm | μm | μm | % < 10μ | Span |
| 3 cm | Actuation 1 | 20.09 | 47.94 | 182 | 1.185 | 3.377 |
| | Actuation 2 | 22.54 | 57.74 | 222.6 | 0.9386 | 3.465 |
| | Actuation 3 | 22.13 | 51.89 | 204.6 | 0.357 | 3.517 |
| | Average | 21.59 | 52.52 | 203.1 | 0.827 | 3.45 |

B. Spray Profile of Fentanyl Spray Formulation #2, at 3 cm

| | | Spray Pattern | | | Plume Geometry | |
|---|---|---|---|---|---|---|
| | | Dmin | Dmax | Ovality | Width | Angle |
| Formulation #2 | | (mm) | (mm) | Ratio | (mm) | (°) |
| 3 cm | Actuation 1 | 21.5 | 42.4 | 1.976 | 20.3 | 19.1 |
| | Actuation 2 | 21.2 | 51.2 | 2.417 | 22.8 | 21.3 |
| | Actuation 3 | 20.5 | 33.2 | 1.622 | 34 | 31.6 |
| | Average | 21.1 | 42.3 | 2.005 | 25.7 | 24.0 |

TABLE 4
A. Particle Size of Fentanyl Spray Formulation #2, at 6 cm

| | | Particle Size | | | | |
|---|---|---|---|---|---|---|
| | | DV(10) | DV(50) | DV(90) | | |
| Formulation #2 | | μm | μm | μm | % <10μ | Span |
| 6 cm | Actuation 1 | 27.38 | 58.55 | 170.5 | 1.216 | 2.445 |
| | Actuation 2 | 26.18 | 57.69 | 178.3 | 1.15 | 2.636 |
| | Actuation 3 | 25.93 | 54.99 | 176.6 | 1.393 | 2.74 |
| | Average | 26.50 | 57.08 | 175.1 | 1.253 | 2.61 |

B. Spray Profile of Fentanyl Spray Formulation #2, at 6 cm

| | | Spray Pattern | | | Plume Geometry | |
|---|---|---|---|---|---|---|
| | | Dmin | Dmax | Ovality | Width | Angle |
| Formulation #2 | | (mm) | (mm) | Ratio | (mm) | (°) |
| 6 cm | Actuation 1 | 21.5 | 42.4 | 1.976 | 20.3 | 19.1 |
| | Actuation 2 | 21.2 | 51.2 | 2.417 | 22.8 | 21.3 |
| | Actuation 3 | 20.5 | 33.2 | 1.622 | 34 | 31.6 |
| | Average | 21.1 | 42.3 | 2.005 | 25.7 | 24.0 |

TABLE 5
A. Particle Size of Fentanyl Spray Formulation #3, at 3 cm

| | | Particle Size | | | | |
|---|---|---|---|---|---|---|
| | | DV(10) | DV(50) | DV(90) | | |
| Formulation #3 | | μm | μm | μm | % < 10μ | Span |
| 3 cm | Actuation 1 | 22.28 | 62.92 | 280.8 | 0.8671 | 4.108 |
| | Actuation 2 | 22.62 | 62.63 | 295.9 | 0.8472 | 4.363 |
| | Actuation 3 | 21.95 | 58.89 | 267.4 | 0.9685 | 4.167 |
| | Average | 22.28 | 61.48 | 281.4 | 0.894 | 4.21 |

B. Spray Profile of Fentanyl Spray Formulation #3, at 3 cm

| | | Spray Pattern | | | Plume Geometry | |
|---|---|---|---|---|---|---|
| | | Dmin | Dmax | Ovality | Width | Angle |
| Formulation #3 | | (mm) | (mm) | Ratio | (mm) | (°) |
| 3 cm | Actuation 1 | 14.4 | 25.5 | 1.775 | 23.8 | 43.3 |
| | Actuation 2 | 16.8 | 24.9 | 1.478 | 23.1 | 42.1 |
| | Actuation 3 | 14 | 25.8 | 1.834 | 22.1 | 40.2 |
| | Average | 15.1 | 25.4 | 1.696 | 23.0 | 41.9 |

TABLE 6
A. Particle Size of Fentanyl Spray Formulation #3, at 6 cm

| | | Particle Size | | | | |
|---|---|---|---|---|---|---|
| | | DV(10) | DV(50) | DV(90) | | |
| Formulation #3 | | μm | μm | μm | % <10μ | Span |
| 6 cm | Actuation 1 | 26.2 | 57.47 | 207.4 | 1.241 | 3.153 |
| | Actuation 2 | 24.82 | 51.65 | 125.7 | 1.48 | 1.954 |
| | Actuation 3 | 28.33 | 51.37 | 114.5 | 0 | 1.678 |
| | Average | 26.45 | 53.50 | 149.2 | 0.907 | 2.26 |

B. Spray Profile of Fentanyl Spray Formulation #3, at 6 cm

| | | Spray Pattern | | | Plume Geometry | |
|---|---|---|---|---|---|---|
| | | Dmin | Dmax | Ovality | Width | Angle |
| Formulation #3 | | (mm) | (mm) | Ratio | (mm) | (°) |
| 6 cm | Actuation 1 | 24.7 | 35.6 | 1.445 | 32.9 | 30.6 |
| | Actuation 2 | 20.3 | 29.7 | 1.46 | 33.6 | 31.3 |
| | Actuation 3 | 20.7 | 31.7 | 1.527 | 27 | 25.1 |
| | Average | 21.9 | 32.3 | 1.477 | 31.2 | 29.0 |

TABLE 7
A. Particle Size of Fentanyl Spray Formulation #4, at 3 cm

| | | Particle Size | | | | |
|---|---|---|---|---|---|---|
| | | DV(10) | DV(50) | DV(90) | | |
| Formulation #4 | | μm | μm | μm | % <10μ | Span |
| 3 cm | Actuation 1 | 20.93 | 57.14 | 242.7 | 1.242 | 3.88 |
| | Actuation 2 | 21.97 | 55.34 | 248.8 | 0.9644 | 4.098 |
| | Actuation 3 | 21.04 | 54.92 | 259.8 | 1.032 | 4.348 |
| | Average | 21.31 | 55.80 | 250.4 | 1.079 | 4.11 |

B. Spray Profile of Fentanyl Spray Formulation #4, at 3 cm

| | | Spray Pattern | | | Plume Geometry | |
|---|---|---|---|---|---|---|
| | | Dmin | Dmax | Ovality | Width | Angle |
| Formulation #4 | | (mm) | (mm) | Ratio | (mm) | (°) |
| 3 cm | Actuation 1 | 16.5 | 28.4 | 1.724 | 25.2 | 44.3 |
| | Actuation 2 | 13.8 | 23.4 | 1.691 | 25.9 | 46.7 |
| | Actuation 3 | 15.8 | 31.1 | 1.963 | 16.8 | 31.3 |
| | Average | 15.4 | 27.6 | 1.793 | 22.6 | 40.8 |

TABLE 8
A. Particle Size of Fentanyl Spray Formulation #4, at 6 cm

| | | Particle Size | | | | |
|---|---|---|---|---|---|---|
| | | DV(10) | DV(50) | DV(90) | | |
| Formulation #4 | | μm | μm | μm | % <10μ | Span |
| 6 cm | Actuation 1 | 26.41 | 54.78 | 204.5 | 1.514 | 3.251 |
| | Actuation 2 | 26.77 | 52.31 | 161.7 | 1.324 | 2.58 |
| | Actuation 3 | 27.04 | 53.54 | 129.6 | 0.9416 | 1.917 |
| | Average | 26.74 | 53.54 | 165.3 | 1.260 | 2.58 |

B. Spray Profile of Fentanyl Spray Formulation #4, at 6 cm

| | | Spray Pattern | | | Plume Geometry | |
|---|---|---|---|---|---|---|
| | | Dmin | Dmax | Ovality | Width | Angle |
| Formulation #4 | | (mm) | (mm) | Ratio | (mm) | (°) |
| 6 cm | Actuation 1 | 19.7 | 30.8 | 1.566 | 38.2 | 34.3 |
| | Actuation 2 | 19.1 | 32.7 | 1.707 | 37.1 | 34.2 |
| | Actuation 3 | 22.4 | 42 | 1.872 | 22.4 | 21.2 |
| | Average | 20.4 | 35.2 | 1.715 | 32.6 | 29.9 |

TABLE 9
A. Particle Size of Fentanyl Spray Formulation #5, at 3 cm

| | | Particle Size | | | | |
|---|---|---|---|---|---|---|
| | | DV(10) | DV(50) | DV(90) | | |
| Formulation #5 | | μm | μm | μm | % <10μ | Span |
| 3 cm | Actuation 1 | 22.03 | 46.84 | 162.9 | 0.519 | 3.007 |
| | Actuation 2 | 21.52 | 57.5 | 266.6 | 1.019 | 4.263 |
| | Actuation 3 | 22.44 | 55.26 | 232.5 | 0.9899 | 3.801 |
| | Average | 22.00 | 53.20 | 220.7 | 0.843 | 3.69 |

B. Spray Profile of Fentanyl Spray Formulation #5, at 3 cm

| | | Spray Pattern | | | Plume Geometry | |
|---|---|---|---|---|---|---|
| | | Dmin | Dmax | Ovality | Width | Angle |
| Formulation #5 | | (mm) | (mm) | Ratio | (mm) | (°) |
| 3 cm | Actuation 1 | 13 | 25 | 1.918 | 17.2 | 31.8 |
| | Actuation 2 | 14.5 | 26.7 | 1.846 | 20.3 | 37.1 |
| | Actuation 3 | 16.7 | 23.3 | 1.398 | 15.8 | 29.4 |
| | Average | 14.7 | 25.0 | 1.721 | 17.8 | 32.8 |

TABLE 10
A. Particle Size of Fentanyl Spray Formulation #5, at 6 cm

| | | Particle Size | | | | |
|---|---|---|---|---|---|---|
| | | DV(10) | DV(50) | DV(90) | | |
| Formulation #5 | | μm | μm | μm | % <10μ | Span |
| 6 cm | Actuation 1 | 25.65 | 56.36 | 248.5 | 1.281 | 3.953 |
| | Actuation 2 | 26.48 | 52.52 | 121.2 | 1.069 | 1.804 |
| | Actuation 3 | 25.08 | 52.25 | 168.3 | 1.419 | 2.741 |
| | Average | 25.74 | 53.71 | 179.3 | 1.256 | 2.83 |

B. Spray Profile of Fentanyl Spray Formulation #5, at 6 cm

| | | Spray Pattern | | | Plume Geometry | |
|---|---|---|---|---|---|---|
| | | Dmin | Dmax | Ovality | Width | Angle |
| Formulation #5 | | (mm) | (mm) | Ratio | (mm) | (°) |
| 6 cm | Actuation 1 | 22.8 | 34 | 1.491 | 24.9 | 23.2 |
| | Actuation 2 | 20.7 | 39.8 | 1.924 | 24.5 | 22.9 |
| | Actuation 3 | 23.2 | 39.8 | 1.712 | 21 | 19.8 |
| | Average | 22.2 | 37.9 | 1.709 | 23.5 | 22.0 |

TABLE 11
A. Particle Size of Fentanyl Spray Formulation #6, at 3 cm

| | | Particle Size | | | | |
|---|---|---|---|---|---|---|
| | | DV(10) | DV(50) | DV(90) | | |
| Formulation #6 | | μm | μm | μm | % <10μ | Span |
| 3 cm | Actuation 1 | 23.63 | 49.01 | 133.7 | 0 | 2.245 |
| | Actuation 2 | 21.55 | 57.08 | 225.4 | 1.059 | 3.571 |
| | Actuation 3 | 21.85 | 60.07 | 262.9 | 0.9586 | 4.012 |
| | Average | 22.34 | 55.39 | 207.3 | 0.673 | 3.28 |

B. Spray Profile of Fentanyl Spray Formulation #6, at 3 cm

| | | Spray Pattern | | | Plume Geometry | |
|---|---|---|---|---|---|---|
| | | Dmin | Dmax | Ovality | Width | |
| Formulation #6 | | (mm) | (mm) | Ratio | (mm) | Angle (°) |
| 3 cm | Actuation 1 | 14.7 | 23.9 | 1.63 | 17.2 | 31.9 |
| | Actuation 2 | 14.9 | 20.9 | 1.396 | 21 | 38.4 |
| | Actuation 3 | 13.7 | 22.9 | 1.671 | 17.9 | 33.1 |
| | Average | 14.4 | 22.6 | 1.566 | 18.7 | 34.5 |

TABLE 12
A. Particle Size of Fentanyl Spray Formulation #6, at 6 cm

| | | Particle Size | | | | |
|---|---|---|---|---|---|---|
| | | DV(10) | DV(50) | DV(90) | | |
| Formulation #6 | | μm | μm | μm | % <10μ | Span |
| 6 cm | Actuation 1 | 25.57 | 52.33 | 157.2 | 1.408 | 2.516 |
| | Actuation 2 | 27.28 | 48.84 | 95.6 | 0 | 1.399 |
| | Actuation 3 | 24.13 | 49.94 | 130.8 | 1.375 | 2.135 |
| | Average | 25.66 | 50.37 | 127.9 | 0.928 | 2.02 |

B. Spray Profile of Fentanyl Spray Formulation #6, at 6 cm

| | | Spray Pattern | | | Plume Geometry | |
|---|---|---|---|---|---|---|
| | | Dmin | Dmax | Ovality | Width | Angle |
| Formulation #6 | | (mm) | (mm) | Ratio | (mm) | (°) |
| 6 cm | Actuation 1 | 21.5 | 36 | 1.673 | 20.7 | 19.5 |
| | Actuation 2 | 21.3 | 34.8 | 1.632 | 37.8 | 35 |
| | Actuation 3 | 22.4 | 32.6 | 1.457 | 24.2 | 22.7 |
| | Average | 21.7 | 34.5 | 1.587 | 27.6 | 25.7 |

### EXAMPLE 4

### Rabbit Pharmacokinetic Data for Fentanyl Formulations

Ten New Zealand white rabbits weighing 2-3 kg were used to determine the pharmacokinetics for sublingual Subsys® formulation. Additionally, five New Zealand white rabbits weighing 2-3 kg were used to evaluate the pharmacokinetics for each novel sublingual fentanyl formulation, in comparison to Subsys® formulation. Before dosing, each rabbit was first anesthetized by Isoflurane gas as needed to keep it immobilized for approximately 15-20 min.

For each formulation, each rabbit in the study received a single dose of 0.1 ml sublingual spray (equivalent to 50 μg of fentanyl base). Specifically, the dose of liquid formulation was administered underneath the tongue using a spray device. Blood samples (approximately 1 ml per sample) were collected through a catheter pre-installed to the ear vein.

Blood samples were collected at pre-dose and 5, 10, 20, 45, 60 and 120 min post-dose. Samples were immediately cooled and plasma was separated by centrifugation within 2-3 hours of blood collection. Samples were stored at −20° C. until assay. After recovering from anesthesia, animals were returned to their cages. These animals were rested in cages for at least 5-7 days before they could be reused for further testing.

The following pharmacokinetic parameters were calculated: peak concentration in plasma (Cₘₐₓ), time to reach Cₘₐₓ (Tₘₐₓ), and area under the concentration-time curve from time-zero to 24 hours postdose (AUC₀₋₂₄ₕ).

At 5 minutes after a single-dose sublingual administration of Subsys® in rabbits, the geometric mean plasma concentration of fentanyl was 1.34 ng/mL. Compared to Subsys®, formulations #2R, #6R, #7R, #8R, #9R and #10R exhibited similar or lower mean plasma concentrations of fentanyl at early time points postdose.

On the contrary, the presence of 0.5% menthol, 0.5% capric acid, or 0.5% caprylic acid to the Subsys® formulation enabled a more rapid absorption of fentanyl. After sublingual administrations of #3R, #4R and #5R, the geometric mean plasma concentrations of fentanyl at 5 minutes postdose were 49%, 54% and 64% higher than that of Subsys®, respectively. In addition, formulations #3R, #4R and #5R have showed Cₘₐₓ values approximately 25%, 44% and 56% higher than Subsys®, respectively. Formulations #3R, #4R and #5R also exhibited AUC₀₋₂₄ₕ values approximately 9%, 4% and 29% higher than Subsys®, respectively.

TABLE 13
Sublingual Fentanyl Formulations for Rabbit Dosing

| | Formulation | | | | | |
|---|---|---|---|---|---|---|
| | #1R | Formulation | Formulation | Formulation | Formulation | Formulation |
| Component | (Subsys ®) | #2R | #3R | #4R | #5R | #6R |
| Dose (ug) | 50 | 50 | 50 | 50 | 50 | 50 |
| Fentanyl Base | 0.0572 | 0.0587 | 0.0584 | 0.0586 | 0.0582 | 0.061 |
| Dehydrated | 54.5 | 54.5 | 54.5 | 54.5 | 54.5 | 54.5 |
| Alcohol | | | | | | |
| Propylene | 4.945 | 4.945 | 4.945 | 4.945 | 4.945 | 4.945 |
| Glycol | | | | | | |
| L-Menthol | 0.05 | 0.05 | 0.05 | 0.05 | 0.5 | 0.5 |
| Xylitol | 2.967 | 2.967 | 2.967 | 2.967 | 2.967 | 2.967 |
| Capric acid | 0 | 0 | 0.5 | 0 | 0 | 0 |
| (C10) | | | | | | |
| Caprylic Acid | 0 | 0 | 0 | 0.5 | 0 | 2 |
| (C8) | | | | | | |
| Sodium | 0 | 0.2 | 0 | 0 | 0 | 0 |
| Deoxycholate | | | | | | |
| Benzalkonium | 0 | 0 | 0 | 0 | 0 | 0.01 |
| Chloride | | | | | | |
| Purified water | 37.4808 | 37.2793 | 36.9796 | 36.9794 | 37.0298 | 35.017 |
| Total | 100 | 100 | 100 | 100 | 100 | 100 |

Values = % w/w

TABLE 14
Additional Sublingual Fentanyl Formulations for Rabbit Dosing

| | Formulation | Formulation | Formulation | Formulation |
|---|---|---|---|---|
| Formulation | #7R | #8R | #9R | #10R |
| Dose (ug) | 50 | 50 | 50 | 50 |
| Fentanyl Base | 0.06 | 0.06 | 0.06 | 0.06 |
| Dehydrated | 55 | 55 | 89.94 | 44.94 |
| Alcohol | | | | |
| Propylene | 5 | 5 | 0 | 45 |
| Glycol | | | | |
| L-Menthol | 5 | 10 | 10 | 10 |
| Xylitol | 3 | 3 | 0 | 0 |
| Purified Water | 31.94 | 26.94 | 0 | 0 |
| Total | 100 | 100 | 100 | 100 |

Values = % w/w

TABLE 15
Geometric mean plasma concentrations for Fentanyl after
sublingual administration of 50 μg single doses of Fentanyl formulations
(see Table 13) to New Zealand white rabbits under fasted conditions.

| | #1R | | | | | |
|---|---|---|---|---|---|---|
| Parameter | (Subsys ®) | #2R | #3R | #4R | #5R | #6R |
| Concentration @ 5 | 1.34 | 1.31 | 2.00 | 2.07 | 2.20 | 1.39 |
| min (ng/mL) | | | | | | |
| Concentration @ 10 | 1.44 | 1.55 | 1.66 | 2.10 | 2.09 | 1.37 |
| min (ng/mL) | | | | | | |
| Concentration @ 15 | 1.47 | 1.35 | 1.59 | 1.89 | 1.96 | 1.24 |
| min (ng/mL) | | | | | | |
| Concentration @ 30 | 1.16 | 1.02 | 1.11 | 1.13 | 1.30 | 0.94 |
| min (ng/mL) | | | | | | |
| Tₘₐₓ (min) | 13 | 10 | 5 | 10 | 5 | 5 |
| Cₘₐₓ (ng/mL) | 1.6 ± 1.6 | 1.6 ± 1.2 | 2.0 ± 1.6 | 2.3 ± 1.4 | 2.5 ± 1.2 | 1.5 ± 1.2 |
| AUC_{0-24 h} | 121.2 ± 1.5 | 119.8 ± | 131.8 ± | 125.8 ± | 156.1 ± | 119.3 ± |
| (ng*min/mL) | | 1.4 | 1.4 | 1.5 | 1.4 | 1.2 |

Tₘₐₓ: median value
Cₘₐₓ and AUC_{0-24 h}: geometric mean ± geometric SD

TABLE 16
Geometric mean plasma concentrations for fentanyl after
sublingual administration of 50 μg single-doses of additional fentanyl
formulations (see Table 14) to New Zealand white rabbits
under fasted conditions.

| Parameter | #7R | #8R | #9R | #10R |
|---|---|---|---|---|
| Concentration | 0.90 | 0.93 | 0.82 | 0.40 |
| @ 5 min | | | | |
| (ng/mL) | | | | |
| Concentration | 1.12 | 1.15 | 0.96 | 0.57 |
| @ 10 min | | | | |
| (ng/mL) | | | | |
| Concentration | 1.16 | 1.35 | 1.00 | 0.64 |
| @ 15 min | | | | |
| (ng/mL) | | | | |
| Concentration | 1.06 | 1.31 | 0.90 | 0.63 |
| @ 30 min | | | | |
| (ng/mL) | | | | |
| Tₘₐₓ (min) | 20 | 20 | 15 | 20 |
| Cₘₐₓ (ng/mL) | 1.3 ± 1.4 | 1.6 ± 1.2 | 1.0 ± 1.2 | 0.7 ± 1.4 |
| AUC_{0-24 h} | 108.7 ± 1.4 | 230.5 ± 1.3 | 137.3 ± 1.3 | 115.0 ± 1.2 |
| (ng * min/mL) | | | | |

Tₘₐₓ: median value
Cₘₐₓ and AUC_{0-24 h}: geometric mean ± geometric SD

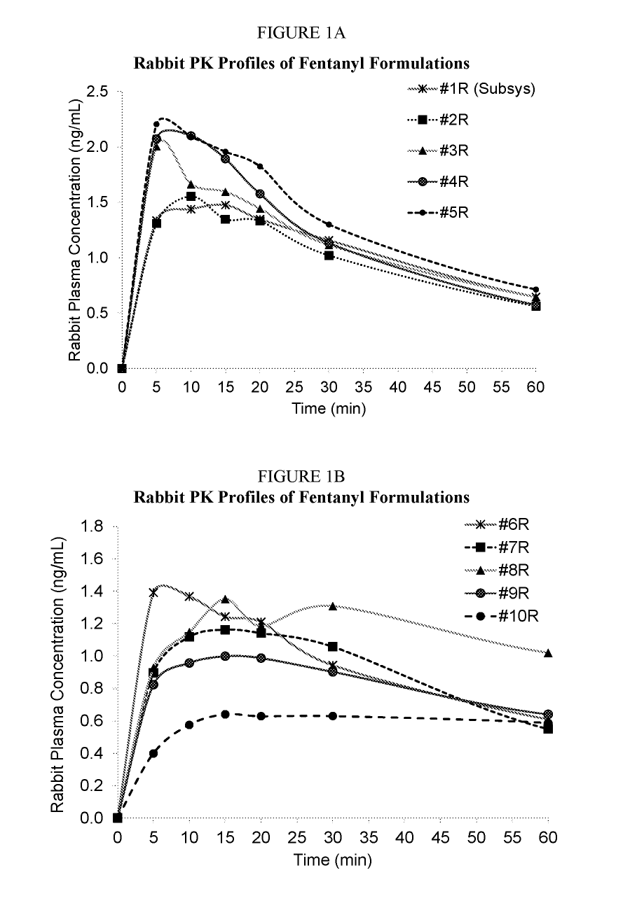

FIGS. 1A and 1B depict pharmacokinetic (PK) profiles of fentanyl compositions 1R through 5R (FIG. 1A) and 6R through 1OR (FIG. 1B).

### EXAMPLE 5

### Mini-Pig Pharmacokinetic Data for Fentanyl Formulations

Protocol design was a single dose crossover study. Four to six healthy male Yucatan mini-pigs weighing approximately forty kilograms each were sublingually administered Subsys® formulation or other novel fentanyl formulations. The mini-pigs were fasted overnight till four hours post administration. Each dosing was followed by a one-week washout period. Blood samples were taken prior to administration and 3, 5, 10, 15, 20, 30 min, 1, 1.5, 2, 3, 4, 5 and 24 hours post dose. Mini-pig plasma samples were measured for fentanyl concentrations via liquid chromatography-tandem mass spectrometry.

The following pharmacokinetic parameters were calculated: peak concentration in plasma (Cₘₐₓ), time to reach Cₘₐₓ (Tₘₐₓ), and area under the concentration-time curve from time-zero to 24 hours postdose (AUC₀₋₂₄ₕ).

The pharmacokinetic behavior of Subsys® and a number of novel fentanyl formulations were evaluated. At 5 minutes after a single-dose sublingual administration of Subsys® in mini-pigs, the geometric mean plasma concentration of Fentanyl was 0.17 ng/mL. Compared to Subsys®, formulations #7M, #8M, #9M and #10M exhibited lower mean plasma concentrations of fentanyl at 5 minutes postdose.

When the menthol concentration in the Subsys® formulation was increased to 0.5%, the resulting formulation #5M enabled a much more rapid absorption of fentanyl. Specifically, #5M showed a geometric mean fentanyl plasma concentration of 0.37 ng/mL at 5 minutes postdose. It was also noted that a plasma concentration of 0.63 ng/mL was achieved as early as 3 minutes after a sublingual administration of #5M. In addition, #5M showed a Cmax increase and an AUC₀₋₂₄ₕ increase of approximately 100% and 33%, compared to Subsys®, respectively.

TABLE 17
Sublingual Fentanyl Formulations for Mini-pig Dosing

| | #1M | | | | | |
|---|---|---|---|---|---|---|
| Formulation | (Subsys ®) | #5M | #7M | #8M | #9M | #10M |
| Dose (ug) | 200 | 200 | 200 | 200 | 200 | 200 |
| Fentanyl Base | 0.235 | 0.232 | 0.237 | 0.239 | 0.262 | 0.235 |
| Dehydrated Alcohol | 54.51 | 54.5 | 54.502 | 54.507 | 89.738 | 44.885 |
| Propylene Glycol | 4.95 | 4.946 | 4.945 | 4.945 | — | 44.88 |
| L-Menthol | 0.05 | 0.5 | 5 | 10.001 | 10 | 10 |
| Xylitol | 2.97 | 2.968 | 2.966 | 2.966 | — | — |
| Purified Water | 37.285 | 36.854 | 32.35 | 27.342 | — | — |
| Total | 100 | 100 | 100 | 100 | 100 | 100 |

Components: % w/w
QS: quantity sufficient
Permeability coefficient: mean ± standard deviation

TABLE 18
Geometric mean plasma concentrations for Fentanyl after
sublingual administration of 200 μg single-doses of Fentanyl formulations
(see Table 17) to Yucatan mini-pigs under fasted conditions.

| | #1M | | | | | |
|---|---|---|---|---|---|---|
| Parameter | (Subsys ®) | #5M | #7M | #8M | #9M | #10M |
| Concentration @ 3 | NC | 0.63 | NC | NC | NC | NC |
| min (ng/mL) | | | | | | |
| Concentration @ 5 | 0.17 | 0.37 | 0.07 | 0.07 | 0.09 | 0.11 |
| min (ng/mL) | | | | | | |
| Concentration @ 10 | 0.21 | 0.43 | 0.24 | 0.26 | 0.21 | 0.26 |
| min (ng/mL) | | | | | | |
| Concentration @ 15 | 0.46 | 0.57 | 0.32 | 0.34 | 0.54 | 0.24 |
| min (ng/mL) | | | | | | |
| Concentration @ 30 | 0.40 | 0.51 | 0.26 | 0.27 | 0.36 | 0.17 |
| min (ng/mL) | | | | | | |
| Tₘₐₓ (min) | 15 | 15 | 20 | 17.5 | 22.5 | 15 |
| Cₘₐₓ (ng/mL) | 0.5 ± 2.0 | 1.0 ± 1.6 | 0.4 ± 1.8 | 0.5 ± 1.8 | 0.6 ± 3.0 | 0.5 ± 2.9 |
| AUC_{0-24 h} | 68.2 ± 1.4 | 90.8 ± 1.6 | 53.0 ± 1.4 | 57.3 ± 1.5 | 67.3 ± 2.2 | 86.8 ± 1.6 |
| (ng*min/mL) | | | | | | |

NC: blood sample not collected
Tₘₐₓ: median value
Cₘₐₓ and AUC_{0-24 h}: geometric mean ± geometric SD

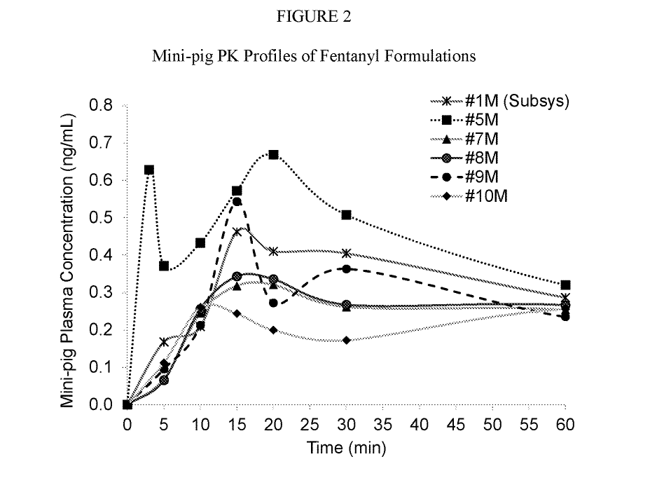

FIG. 2 depicts pharmacokinetic (PK) profiles of fentanyl compositions 1M, 5M, 7M, 8M, 9M and 10M.

### EXAMPLE 6

### In Vitro Permeability of Fentanyl Sublingual Spray Formulations Across Human Skin

In vitro permeability of fentanyl formulations across human skin was evaluated to identify the formulations with better permeability and flux. Franz diffusion apparatus and epidermal layer of the human skin were used to evaluate permeability.

Heat separation method was used to separate the epidermis from the skin. Specifically, a previously cut (1 in×1 in) skin piece was placed into 65° C. phosphate buffered saline (PBS, pH 7.34) for two minutes, and then promptly removed and placed in a clear petri dish with a minimal amount of PBS. Afterwards, the fatty dermis layer was firmly held in place with a spatula and gently picked at the upper epidermis until it begins to separate. Upon noticeable separation, the full edge can be separated with a gentle practice across the tissue. Finally, the entire epidermis was pulled up and away from the lower dermis in small increments. Each epidermal membrane was placed between the 9 mm donor chamber and the receptor cell, and secured with clamps.

At the beginning of the study (i.e., time zero), 5 mL PBS was used as receiver media and 0.5 mL of test formulation was loaded to the donor chamber. The membrane integrity was evaluated by the handheld Keysight LCR meter U1731C.

Subsequently, 0.2 mL of the sample was collected from the receptor cell at predetermined time intervals and replaced with 0.2 mL of fresh PBS immediately. Collected samples were filtered with 0.45 μm Nylon membrane and then analyzed using an HPLC method.

In vitro flux and permeability coefficient of fentanyl across human skin (Subsys® or #F1; control formulation) were 0.13 μg/min/sec² and 5.38×10⁻⁷ cm/sec. With an increase in the menthol concentration to 5% or 10%, both flux and permeability coefficients of fentanyl were decreased (#F2, #F3, #F4 and #F5). In contrast, with an increase of menthol concentration to 0.5 and 1%, permeability coefficients were increased by 3-fold (i.e., 15.7×10⁻⁷ cm/sec for #F6) and 2-fold (i.e., 9.19×10⁻⁷ cm/sec for #F7), respectively.

When permeation enhancers such as oleic acid (#F8), sodium lauryl sulfate (#F9), sodium deoxycholate (#F10), and cetylpyridinium chloride (#F11) were used in the formulation, greater permeability coefficient of fentanyl was achieved compared to the control formulation (#F1). Use of capric acid (0.5% w/w) as permeation enhancer gave the highest flux and permeability coefficient with a value of 0.53 μg/min/sec² and 22.2×10⁻⁷ cm/sec, respectively.

In addition, inclusion of both 0.5% menthol and 0.5% capric acid (#F14) was evaluated for permeability of fentanyl across human skin. It was observed that the combination also improved the permeability coefficient of fentanyl, compared to Subsys®.

Based on the result, it was concluded that presence of menthol and capric acid at 0.5% has the highest possibility to enhance the permeability of fentanyl across sublingual mucosa in vivo.

Table 19 below lists the ingredients of each tested fentanyl formulation, as well as the measured flux and permeability coefficient values.

TABLE 19
In vitro human skin permeability of fentanyl sublingual spray
formulations at different concentrations of menthol as permeation enhancer.

| Formulation | #F1 | | | | | | |
|---|---|---|---|---|---|---|---|
| # | (Subsys ®) | #F2 | #F3 | #F4 | #F5 | #F6 | #F7 |
| Fentanyl | 0.4395 | 0.4395 | 0.4395 | 0.4395 | 0.4395 | 0.4395 | 0.4395 |
| Base | | | | | | | |
| Dehydrated | 54.5 | 54.5 | 54.5 | Q.S | 43.26 | 54.5 | 54.5 |
| Alcohol | | | | | | | |
| Propylene | 4.945 | 4.945 | 4.945 | 4.945 | 43.26 | 4.945 | 4.945 |
| Glycol | | | | | | | |
| L-Menthol | 0.05 | 5 | 10 | 10 | 10 | 0.5 | 1 |
| Xylitol | 2.967 | 2.967 | 2.967 | 2.967 | 2.967 | 2.967 | 2.967 |
| Purified | QS | QS | QS | — | — | QS | QS |
| water | | | | | | | |
| Total | 100 | 100 | 100 | 100 | 100 | 100 | 100 |
| Flux | 0.13 | 0.10 | 0.06 | 0.03 | 0.03 | 0.38 | 0.22 |
| (μg/min/cm²) | | | | | | | |
| Permeability | 5.38 ± | 4.37 ± | 2.71 ± | 1.53 ± | 1.37 ± | 15.7 ± | 9.19 ± |
| coefficient | 1.22 | 0.42 | 0.81 | 0.17 | 0.07 | 2.62 | 1.49 |
| (×10⁻⁷ cm/sec) | | | | | | | |

Components: % w/w
QS: quantity sufficient
Permeability coefficient: mean ± standard deviation

Table 20 describes In vitro human skin permeability of fentanyl sublingual spray formulations with different permeation enhancers at different concentrations.

TABLE 20
In vitro human skin permeability of fentanyl sublingual spray
formulations with different permeation enhancers at different concentrations.

| Formulation | #F8 | #F9 | #F10 | #F11 | #F12 | #F13 |
|---|---|---|---|---|---|---|
| Fentanyl Base | 0.4395 | 0.4395 | 0.4395 | 0.4395 | 0.4395 | 0.4395 |
| Dehydrated | 54.5 | 54.5 | 54.5 | 54.5 | 54.5 | 54.5 |
| Alcohol | | | | | | |
| Propylene | 4.945 | 4.945 | 4.945 | 4.945 | 4.945 | 4.945 |
| Glycol | | | | | | |
| L-Menthol | 0.05 | 0.05 | 0.05 | 0.05 | 0.05 | 0.05 |
| Oleic acid | 0.2 | — | — | — | — | — |
| Sodium lauryl | — | 0.2 | — | — | — | — |
| sulfate | | | | | | |
| Sodium | — | — | 0.2 | — | — | — |
| deoxycholate | | | | | | |
| Cetylpyridinium | — | — | — | 0.2 | — | — |
| chloride | | | | | | |
| Capric acid | — | — | — | — | 0.5 | — |
| (C10) | | | | | | |
| Caprylic acid | — | — | — | — | — | 0.5 |
| (C8) | | | | | | |
| Xylitol | 2.967 | 2.967 | 2.967 | 2.967 | 2.967 | 2.967 |
| Purified water | QS | QS | QS | QS | QS | QS |
| Total | 100 | 100 | 100 | 100 | 100 | 100 |
| Flux | 0.27 | 0.29 | 0.47 | 0.26 | 0.53 | 0.40 |
| (μg/min/cm²) | | | | | | |
| Permeability | 11.1 ± 1.49 | 12.0 ± 4.07 | 19.5 ± 6.11 | 11.0 ± 1.73 | 22.2 ± 6.83 | 16.7 ± 3.02 |
| coefficient | | | | | | |
| (×10⁻⁷ cm/sec) | | | | | | |

Components: % w/w
QS: quantity sufficient
Permeability coefficient: mean ± standard deviation

Table 21 depicts in vitro human skin permeability of fentanyl sublingual spray formulations with one or more permeation enhancers and different concentrations.

TABLE 21
In vitro human skin permeability of fentanyl sublingual spray
formulations with one or more permeation enhancers and different
concentrations.

| | Formulation | | | |
|---|---|---|---|---|
| | #F14 | #F15 | #F16 | #F17 |
| Fentanyl Base | 0.4395 | 0.4395 | 0.4395 | 0.4395 |
| Dehydrated | 54.5 | 54.5 | 20 | 54.5 |
| Alcohol | | | | |
| Propylene | 4.945 | 4.945 | | 4.945 |
| Glycol | | | | |
| L-Menthol | 0.5 | 0.05 | | 0.05 |
| Capric acid | 0.5 | 0.5 | — | — |
| (C10) | | | | |
| Tween 80 | — | 0.1 | — | — |
| Miglyol 812N | — | — | QS | — |
| Citric acid | — | — | — | 0.1 |
| Sodium citrate | — | — | — | 0.1 |
| Xylitol | 2.967 | 2.967 | | 2.967 |
| Purified water | QS | QS | — | QS |
| Total | 100 | 100 | 100 | 100 |
| Flux | 0.37 | 0.29 | 0.16 | 0.19 |
| (μg/min/cm²) | | | | |
| Permeability | 15.4 ± 1.68 | 12.1 ± 2.05 | 6.86 | 7.84 ± 2.02 |
| coefficient | | | (standard | |
| (×10⁻⁷ cm/sec) | | | deviation not | |
| | | | available) | |

Components: % w/w
QS: quantity sufficient
Permeability coefficient: mean ± standard deviation

What is claimed is:

1. A sublingual solution formulation comprising fentanyl or a pharmaceutically acceptable salt or ester thereof, ethanol, propylene glycol and a permeation enhancer, wherein the permeation enhancer is selected from the group consisting of: a) menthol at a concentration from 0.25% to 10% w/w; and b) a fatty acid at a concentration from 0.25% to 10% w/w, wherein w/w refers to weight by total weight of the formulation.

2. The formulation of claim 1, wherein the formulation is a liquid spray formulation.

3. The formulation of claim 1, wherein the permeation enhancer is menthol.

4. The formulation of claim 1, wherein the fatty acid is capric acid or caprylic acid.

5. The formulation of claim 1 wherein fentanyl or the pharmaceutically acceptable salt or ester thereof is present at a concentration from 0.1% to 2% w/w.

6. The formulation of claim 5 wherein fentanyl or the pharmaceutically acceptable salt or ester thereof is present at a concentration from 0.3% to 0.7% w/w

7. The formulation of claim 1 wherein propylene glycol is present at a concentration from 1% to 10% w/w.

8. The formulation of claim 7 wherein propylene glycol is present at a concentration from 4% to 6% w/w.

9. The formulation of claim 1 wherein ethanol is present at a concentration from 40% to 60% w/w.

10. The formulation of claim 1 wherein the concentration of menthol is from 0.25% to 10% w/w.

11. The formulation of claim 10 wherein the concentration of menthol is from 0.25% to 1% w/w.

12. The formulation of claim 4 wherein the concentration of capric acid or caprylic acid is from 0.25% to 10% w/w.

13. The formulation of claim 12 wherein the concentration of capric acid or caprylic acid is from 0.25% to 1% w/w.

14. The formulation of claim 1 further comprising xylitol.

15. The formulation of claim 1 wherein said formulation has a human skin permeability coefficient of greater than 7×10⁻⁷ cm/sec.

16. A sublingual spray formulation comprising fentanyl or a pharmaceutically acceptable salt or ester thereof at a concentration from 0.1% to 0.8% w/w, ethanol at a concentration from 40% to 60% w/w, propylene glycol at a concentration from 4% to 6% w/w, and capric acid at a concentration from 0.25% to 10% w/w, wherein w/w refers to weight by total weight of the formulation.

17. The formulation of claim 16, wherein said formulation further comprises menthol.

18. A sublingual spray formulation comprising fentanyl or a pharmaceutically acceptable salt or ester thereof at a concentration from 0.1% to 0.8% w/w, ethanol at a concentration from 40% to 60% w/w, propylene glycol at a concentration from 4% to 6% w/w, and caprylic acid at a concentration from 0.25% to 10% w/w, wherein w/w refers to weight by total weight of the formulation.

19. The formulation of claim 19, wherein said formulation further comprises menthol.

20. A method of treating pain comprising administering to a patient in need thereof a therapeutically effective amount of a formulation of claim 1.
